(19) United States
(12) Patent Application Publication
(10) Pub. No.: US 2025/0326703 A1
(43) Pub. Date: Oct. 23, 2025

(54) HIGH PURITY FLUOROOLEFIN COMPOSITIONS AND METHODS OF IMPURITY REMOVAL

(71) Applicant: THE CHEMOURS COMPANY FC, LLC, WILMINGTON, DE (US)

(72) Inventors: SHENG PENG, HOCKESSIN, DE (US); ALLEN CAPRON SIEVERT, ELKTON, MD (US); PAUL DOUGLAS BROTHERS, CHADDS FORD, PA (US)

(73) Assignee: THE CHEMOURS COMPANY FC, LLC, WILMINGTON, DE (US)

(21) Appl. No.: 18/864,767

(22) PCT Filed: May 22, 2023
(86) PCT No.: PCT/US2023/023051

§ 371 Date: Nov. 11, 2024

Related U.S. Application Data
(60) Provisional application No. 63/344,859, filed on May 23, 2022.

(51) Int. Cl. C07C 17/389; C07C 21/18; C09K 5/04

(52) U.S. Cl. CPC C07C 17/389; C07C 21/18; C09K 5/045; C09K 2205/122

(57) ABSTRACT

The present invention relates methods of producing, blending, charging, replacing, and packaging fluoroolefin-containing compositions free of oxygen and oxidizing components and/or free of oligomer inhibitors and/or moisture.

### CROSS-REFERENCE TO RELATED APPLICATIONS

This application claims priority under 35 U.S.C. 119 (e) to U.S. Provisional Application No. 63/344,859, filed May 23, 2022, entitled “HIGH PURITY FLUOROOLEFIN COMPOSITIONS-IMPURITY REMOVAL,” the entire contents of which are hereby incorporated by reference.

### BACKGROUND OF THE INVENTION

### Field of the Invention

The present invention relates to high purity HFO and/or HFC containing components and blends which are substantially free of oxygen or oxidizing components (with or without oligomer or polymer inhibitors), and methods of producing, blending, charging, replacing, and packaging said components and blends.

### DESCRIPTION OF RELATED ART

Tetrafluoropropenes, such as HFO-1234yf and HFO-1234ze, are stable molecules under refrigeration usage conditions, as are HFC-125, HFC-134a, HFC-134, HFC-23, HCFOs such as E-HCFO-1233zd, Z-HCFO-1233zd, and HFOs such as E-HFO-1336mzz and Z-HFO-1336mzz, any of which may be blended with HFO-1234yf and/or HFO-Z/E-1234ze and which are used as heat transfer/refrigerant materials. However, fluoroolefins, such as HFO-1234yf can oligomerize or homopolymerize in the presence of air during use, during storage, and/or under severe conditions. One solution is to use inhibitors like terpenes, terpenoids, terpinenes, linear unsaturated hydrocarbons, and phenolic compounds as disclosed in each of U.S. Patent Publication Nos. US20210108119 and US20210040368, each disclosure of which is hereby incorporated herein by reference in its entirety. In addition, ethane, propane, cyclopropane, propylene, butane, butene, isobutane, isobutene, 2-methylbutane, meta-, ortho- or para-xylene, alpha (α)-methyl styrene, 2-methyl-alpha-methylstyrene (a, 2-dimethylstyrene), 3-methyl-alpha-methylstyrene (a, 3-dimethylstyrene) and 4-methyl-alpha-methylstyrene (α, 4-dimethylstyrene), or mixtures of two or more thereof, in effective amounts, generally less than 0.5 weight percent, also inhibit oligomerization or homopolymerization as disclosed, for example, in U.S. Provisional Application No. 63/321,118, filed on Mar. 18, 2022, and entitled “HYDROCARBON ADDITIVES FOR 1234YF AND HFC COMPOSITIONS, METHODS FOR THEIR PRODUCTION, STORAGE AND USAGE”, and 63/321,120, filed on Mar. 18, 2022, and entitled “HYDROCARBON ADDITIVES FOR 1234YF COMPOSITION AND METHODS FOR THEIR PRODUCTION, STORAGE AND USAGE”, each disclosure of which is hereby incorporated by reference in its entirety. The inhibitors disclosed in U.S. Patent Publication Nos. US20210108119 and US20210040368 effectively react with oxygen and/or function as chain transfer agents to terminate the oligomer/polymer chain propagation and limit the amount of oligomer/polymer by-products to at most 0.03 wt. % or 300 ppm, or at most 200 ppm, or preferably at most 100 ppm. However, the presence of the inhibitor may also reduce the purity of the refrigerant going to the refrigerant system. Therefore, it is desirable to provide high purity HFO and/or HFC containing compositions by removing oxygen contaminants and moisture, as well as any oligomer or polymer inhibitors (sometimes referred to herein collectively as “inhibitor” or “inhibitors”) from HFO-1234yf and/or HFO-Z/E-1234ze, or other HFO, HCFO, HFC, HCC, HCFC, and carbon dioxide (CO₂) components, specifically HFC-32, HFC-125, HFC-134a, HFC-134, HFC-23, HFC-227ea, HFC-152a, E-HCFO-1233zd, Z-HCFO-1233zd, E-HFO-1336mzz and Z-HFO-1336mzz, HFO-1132a, and HFO-Z/E-1132 components, or any other additional refrigerant component, alone or in blends, prior to or during loading, blending, reclaiming and packing of these heat transfer/refrigerant materials.

### SUMMARY OF THE INVENTION

Disclosed herein are high purity, substantially oxygen-free fluoroolefin containing compositions including at least 2,3,3,3-tetrafluoropropene, and methods of producing, providing, blending, charging, replacing, and packaging the high purity substantially oxygen-free 2,3,3,3-tetrafluoropropene containing heat transfer/refrigerant compositions and feeds.

Disclosed herein are methods for reducing the level of oxygen in fluoroolefin containing compositions including at least 2,3,3,3-tetrafluoropropene to levels below about 10 ppm, or to levels below about 5 ppm.

Disclosed herein are high purity, substantially oxygen-free 1,3,3,3-tetrafluoropropene ((E/Z)-HFO-1234ze), HFC-32, HFC-125, HFC-134a, HFC-134, HFC-23, E-HCFO-1233zd, Z-HCFO-1233zd, E-HFO-1336mzz and Z-HFO-1336mzz components, compositions and blends and methods of producing, blending and packaging these components, compositions and blends.

Disclosed herein are techniques which determine, remove/eliminate, or reduce/minimize contaminants (e.g., oxygen, oxidizing compounds, and/or oligomer- or polymer-inhibiting agents) present in fluoroolefin containing compositions from stored heat transfer/refrigerant compositions, e.g., tanks, containers, or cannisters, filled with at least one HFO component, alone or in blends with other HFO, HCFO, HFC, HCC, or HCFC components, whether fresh, reclaimed or unreclaimed/used/spent.

Disclosed herein are techniques which improve the stability of high purity fluoroolefin containing compositions by analyzing unstabilized neat or oligomer/polymer-inhibiting stabilized HFO, HCFO, HFC, HCC, or HCFC components, compositions or blends, optionally including carbon dioxide (CO₂), contacting the components or compositions with compounds that can effectively reduce the oxygen level of the unstabilized neat or oligomer/polymer-inhibiting stabilized HFO, HFCO, HFC, HCC, or HCFC components, compositions or blends to at most about 10 ppm, preferably at most about 5 ppm, and optionally analyzing the substantially oxygen-free components, compositions and blends. The source of components, compositions or blends include, but are not limited to, process streams, refrigerant circuits and equipment, and storage vessels, e.g., tanks, containers or cannisters.

Disclosed herein are high purity, substantially inhibitor-free and optionally moisture-free fluoroolefin containing components, compositions and blends including at least 2,3,3,3-tetrafluoropropene, and methods of producing such components, compositions and blends, and using the high purity, substantially inhibitor-free and optionally moisture-free, fluoroolefin containing components, compositions and blends in refrigerant/heat transfer equipment.

Also disclosed are high purity, substantially moisture-, oligomer/polymer inhibitor-, and/or oxygen-free fluoroolefin containing compositions, and methods of producing and charging of refrigerant equipment with the high purity, substantially moisture-, oligomer/polymer inhibitor-, and/or oxygen-free fluoroolefin containing compositions.

Also disclosed herein are methods for contacting fluoroolefin containing compositions including at least one HFO, HCFO, HFC, HCC and HCFC component with a reduced metal oxide compound capable of removing or eliminating at least one of oxygen and oxidizing compounds.

Disclosed herein are methods for contacting fluoroolefin containing compositions including at least 2,3,3,3-tetrafluoropropene with a compound adapted to remove at least one of oxygen and oxidizing compounds without cleavage of the C—F bond connected to the double bond of 2,3,3,3-tetrafluoropropene.

Disclosed herein are methods for contacting fluoroolefin containing compositions including at least 2,3,3,3-tetrafluoropropene with a compound suitable for removing the oligomer-inhibiting or polymer-inhibiting agent in the stabilized fluoroolefin containing compositions including at least 2,3,3,3-tetrafluoropropene, as well as eliminating or reducing moisture.

Disclosed herein are methods of blending substantially oxygen-free fluoroolefin containing compositions including at least 2,3,3,3-tetrafluoropropene, with at least one of 1,3,3,3-tetrafluoropropene (HFO-Z/E-1234ze), HFC-32, HFC-125, HFC-134a, HFC-134, HFC-23, HFC-227ea, HFO-Z/E-1132, HFO-1132a, E-HCFO-1233zd, Z-HCFO-1233zd, E-HFO-1336mzz and Z-HFO-1336mzz, which is also substantially oxygen-free.

Disclosed herein are storage vessels, tanks, and cannisters filled with substantially oxygen-free 2,3,3,3-tetrafluoropropene, alone or combined with at least one additional substantially oxygen-free component selected from 1,3,3,3-tetrafluoropropene (HFO-Z/E-1234ze), HFC-32, HFC-125, HFC-134a, HFC-134, HFC-23, HFC-227ea, HFO-Z/E-1132, HFO-1132a, E-HCFO-1233zd, Z-HCFO-1233zd, E-HFO-1336mzz and Z-HFO-1336mzz.

Disclosed herein are storage vessels, tanks and cannisters filled with substantially oxygen-free 2,3,3,3-tetrafluoropropene, alone or combined with HFO-Z/E-1234ze, optionally containing at least one additional substantially oxygen-free component selected from HFC-32, HFC-125, HFC-134a, HFC-134, HFC-23, HFC-227ea, E-HCFO-1233zd, Z-HCFO-1233zd, E-HFO-1336mzz and Z-HFO-1336mzz under pressure.

Disclosed herein are methods of preparing and charging refrigerant equipment with high purity, substantially moisture-free, oligomer/polymer inhibitor-, and oxygen-free fluoroolefin containing compositions.

Disclosed herein are methods of preparing and charging refrigerant equipment with high purity, substantially moisture- and oligomer/polymer inhibitor-free fluoroolefin containing compositions.

Disclosed herein are also methods of producing high purity fluoroolefin containing compositions by contacting an inhibitor-stabilized fluoroolefin composition with an absorbent bed to remove the inhibitors and their oxidation products. The inhibitor-free fluoroolefin composition may be charged to a refrigerant system or further treated with a reduced metal oxide to reduce or remove oxygen and/or oxidizing compounds to form high purity fluoroolefins suitable for producing, blending, charging, replacing, and packaging substantially oxygen-free fluoroolefin, e.g., HFO-1234yf, containing compositions. Absorbents such as silica gel or mineral oil can be used to remove or scrub the inhibitors, especially terpene, and their oxidation products to ensure that high purity HFO-1234yf goes to the refrigerant equipment and/or system, and other materials such as molecular sieves, carbon, activated carbon, alumina, diatomaceous earth may be used.

Disclosed herein are techniques which improve the stability of high purity fluoroolefin containing compositions by removing/eliminating or reducing contaminants e.g., oxygen and oxidizing compounds or oligomer-inhibiting or polymer-inhibiting agents. The supply of fluoroolefin containing compositions to be processed, includes, but is not limited to refrigerant equipment, a refrigerant circuit, storage vessels, e.g., container, tanks, or cannisters, filled with at least one high purity HFO and HCFO, alone or in blends, which subsequently contacts a reduced metal oxide to reduce or eliminate oxygen present in the high purity fluoroolefin containing compositions.

Disclosed herein are techniques which remove/eliminate or reduce contaminants, e.g., oxygen and oxidizing compounds or oligomer-inhibiting or polymer-inhibiting agents, in fluoroolefin containing compositions collected in storage vessels, e.g., containers, tanks, cannisters, filled with at least one HFO and HFCO compound, alone or in blends with HFO, HFCO, HFC, HCC, HCFC to be recycled and/or reclaimed.

Disclosed herein are techniques which improve the stability of stabilized fluoroolefin containing compositions by contacting the stabilized fluoroolefin containing compositions, e.g., HFO-1234yf alone or combined with HFO-Z/E-1234ze, optionally containing at least one of HFC-125, HFC-134a, HFC-134, HCC-23, HFC-227ea, HFO-Z/E-1132, HFO-1132a, HCFO-Z/E-1233zd, and HFO-Z/E-1336mzz with a reduced metal oxide to reduce or eliminate oxygen.

Disclosed herein are processes for contacting a fluoroolefin-containing composition, e.g., HFO-1234yf alone or combined with HFO-Z/E-1234ze, with a reduced metal oxide to reduce or eliminate oxygen, and optionally blending the substantially oxygen-free HFO-1234yf or HFO-1234yf/-Z/E-1234ze blend with at least one of HFC-125, HFC-134a, HFC-134, HFC-23, HFC-227ea, HFO-Z/E-1132, HFO-1132a, E-HCFO-1233zd, Z-HCFO-1233zd, E-HFO-1336mzz and Z-HFO-1336mzz.

Disclosed herein are processes for (1) contacting a fluoroolefin-containing composition, e.g., HFO-1234yf alone or combined with HFO-Z/E-1234ze, with a reduced metal oxide to reduce or eliminate oxygen and form a substantially oxygen-free stream of HFO-1234yf alone or combined with HFO-Z/E-1234ze; (2) contacting at least one of HFC-125, HFC-134a, HFC-134, HFC-23, HFC-227ea, E-HCFO-1233zd, Z-HCFO-1233zd, E-HFO-1336mzz and Z-HFO-1336mzz with a reduced metal oxide to reduce or eliminate oxygen and form a substantially oxygen-free stream of at least one of HFC-125, HFC-134a, HFC-134, HFC-23, HFC-227ea, HCFO-Z/E-1233zd, HFO-Z/E-1132, HFO-1132a, and HFO-Z/E-1336mzz; and (3) packaging the substantially oxygen-free HFO-1234yf alone and/or with the substantially oxygen-free HFO-Z/E-1234ze and/or blends of (1); or blends of (1) and (2).

Disclosed herein are techniques which improve the stability of fluoroolefin containing compositions by contacting an unstabilized or stabilized supply of HFO-1234yf alone or combined with at least one of HFC-125, HFC-134a, HFC-134, HFC-23, HCFO-Z/E-1233zd, and HFO-Z/E-1336mzz which may be from a high purity or reclaimed source (‘reclaimed’ refrigerant derived from treating a spent/used refrigerant to remove impurities and/or reconstituted to be ASHRAE compliant). The source includes, but is not limited to, storage vessels, e.g., container, tanks, or cannisters, filled with (1) at least HFO-1234yf and HFO-Z/E-1234ze alone or combined with at least one of HFC-125, HFC-134a, HFC-134, HFC-23, HFO-Z/E-1132, HFO-1132a, HFO-Z/E-1233zd, and HFO-Z/E-1336mzz which may be from a high purity or reclaimed source, each stabilized or unstabilized, with at least one inhibitor which effectively reacts with oxygen and/or act as chain transfer agents to terminate the oligomer/polymer chain propagation of HFO-1234yf.

Disclosed herein are techniques which improve the stability of fluoroolefin containing compositions by contacting an unstabilized or stabilized supply of HFO-1234yf and HFO-Z/E-1234ze alone or combined with at least one of HFC-32, HFC-125, HFC-134a, HFC-134, HFC-23, HFC-227ea, HFO-Z/E-1132, HFO-1132a, HCFO-Z/E-1233zd, and HFO-Z/E-1336mzz which may be from a high purity (fresh source) or a reclaimed source (‘reclaimed’ refrigerant derived from treating a spent refrigerant to remove impurities and/or reconstituted to be ASHRAE compliant). The source includes, but is not limited to, storage vessels, e.g., container, tanks, or cannisters filled with (1) at least HFO-1234yf and HFO-Z/E-1234ze alone or combined with at least one of HFC-125, HFC-134a, HFC-134, HFC-23, HCFO-Z/E-1233zd, and HFO-Z/E-1336mzz.

Disclosed herein are systems and methods of using oxygen removal columns which contain a zero valent or low valent transition metal or reduced transition metal oxide which can effectively reduce the oxygen level below about 10 ppm to about 0 ppm, and all values therebetween. The transition metals include Cu, Ti, V, Mn, Fe, Co, Zn, Ni, and Pd and oxygen removal proceeds without C—F bond cleavage of at least the HFO-1234yf fluoroolefin component of the composition, for loading, charging, blending, and packing of the products.

In one embodiment, 2,3,3,3-tetrafluoropropene (HFO-1234yf) alone or combined with (Z/E)-1,1,1,3-tetrafluoropropene (HFO-Z/E-1234ze, R-1234ze or 1234ze), containing from 0 to 100 weight percent HFO-1234yf and from 0.01 to 99.9 weight percent HFO-Z/E-1234ze, is passed through a bed of silica or other suitable materials such as molecular sieves, carbon, activated carbon, alumina, diatomaceous earth, etc. to remove or reduce moisture and any oligomer or polymer inhibitor, including, but not limited to, d-Limonene and α-terpinene, 2-methylbutane, meta-, ortho- or para-xylene, alpha (α)-methyl styrene, 2-methyl-alpha-methylstyrene (α, 2-dimethylstyrene), 3-methyl-alpha-methylstyrene (a, 3-dimethylstyrene) and 4-methyl-alpha-methylstyrene (a, 4-dimethylstyrene).

In another embodiment, HFO-1234yf alone or combined with HFO-Z/E-1234ze, optionally containing at least one of HFC-125, HFC-134a, HFC-134, HFC-23, HFC-227ea, HFO-Z/E-1132, HFO-1132a, HCFO-Z/E-1233zd, and HFO-Z/E-1336mzz is passed through a bed of silica to remove or reduce moisture and oligomer/polymer inhibitor followed by an oxygen removal column.

In one embodiment, 2,3,3,3-tetrafluoropropene (HFO-1234yf) alone or combined with (Z/E)-1,1,1,3-tetrafluoropropene (HFO-Z/E-1234ze, R-1234ze or 1234ze), containing from greater than 0 to 100 weight percent HFO-1234yf and from 0.01 to 99.9 weight percent HFO-Z/E-1234ze, contacts a reduced metal oxide in a treatment zone at ambient temperature to reduce the oxygen content to about 10 ppm or less, preferably to about 5 ppm or less, including to 0 ppm and all values and ranges therebetween.

### DETAILED DESCRIPTION OF THE INVENTION

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed provided that these additional included materials, steps, features, components, or elements do materially affect the basic and novel characteristic(s) of the claimed invention, especially the mode of action to achieve the desired result of any of the processes of the present invention. The term ‘consisting essentially of’ occupies a middle ground between “comprising” and ‘consisting of.’

Where applicants have defined an invention or a portion thereof with an open-ended term such as “comprising,” it should be readily understood that (unless otherwise stated) the description should be interpreted to also include such an invention using the terms “consisting essentially of” or “consisting of.”

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise. The AHRI 2019 Standard for Specifications for Refrigerants sets the maximum level of air and non-condensable components for single refrigerants, e.g., R-12, R-13, R-22, R-23, R-32, R-114, R-115, R-116, R-124, R-125, R-134a, R-142b (2 vol %), R-143a, R-152a, R-218, R-227ea, R236fa, R1234yf, and R-1234ze (E), e.g., at about 1.5 volume percent at 25° C., which would equate to approximately 0.315 volume percent for oxygen (˜21% of air), or approximately 3150 ppm. Notwithstanding the AHRI standards, such high oxygen content leads to oligomerization/polymerization of HFO-1234yf and other fluoroolefins during storage and use, where the oxygen content can also increase during storage, charging, use, recycle and reuse.

The present invention relates to products and methods for producing high purity HFO-1234yf fluoroolefin containing compositions and blends where the oxygen content is at most 5 ppm, 6 ppm, 7 ppm, 8 ppm, 9 ppm or 10 ppm. to avoid the oligomerization/polymerization of at least the HFO-1234yf component. Oxygen is similarly removed from any additional HFO, HCFO, HFC, HCC, or HCFC component that is to be blended with HFO-1234yf and/or HFO-Z/E-1234ze, even those refrigerants where AHRI does not set the non-condensable limit at 1.5 volume percent.

Oxygen contaminants are removed by contacting a stream of the HFO-1234yf and/or the HFO-Z/E-1234ze components, or any additional HFO, HCFO, HFC, HCC, or HCFC component to be blended, with a compound that removes the oxygen contaminant without cleavage of a C—F bond in the fluoroolefin such as 2,3,3,3-tetrafluoropropene (HFO-1234yf).

Deoxygenation reagents suitable for removing or reducing oxygen from fluoroolefin-containing mixtures at room temperature, e.g., 20° to 30° C., include, but are not limited to, zero valent or low valent transition metals, and reduced oxides of transitions metals where the transition metal is selected from Cu, Ti, V, Mn, Fe, Co, Zn, Ni, and Pd. Zero valent or low valent transition metals and reduced oxides of transitions metals may be obtained commercially or may be prepared by heating a transition metal oxide in the presence of a reducing agent, such as hydrogen, at an elevated temperature (e.g., 100° C. to 300° C.) in a vessel such as a tube or column. Examples of metal oxides suitable for reduction in this manner include CuO, TiO₂, V₂O₅, Mn₂O₃, Fe₂O₃, Co₃O₄, ZnO, NiO, and PdO. Other deoxygenation reagents include solutions of ascorbic acid salts, NaHSO₃, Na₂SO₃, Na₂S₂O₅, Na₂S₂O₃, Na₂S₂O₄, polyhydroxy benzene compounds such as pyrogallol; Ti (III) salts, Cr (II) salts, Sn (II) salts, Fe (II) salts; nitrite salts, and hypophosphite salts. Oxygen removal from fluoroolefin compositions may be conducted by passing the fluoroolefin composition through a column or bed of deoxygenation reagent at 20° C. to 30° C. In embodiments where oxygen removal is affected by solutions of ascorbic acid salts, the treated fluoroolefin containing composition is preferably dried by passage through a bed or tube containing a desiccant, such as CaSO₄, CaCl₂), Na₂SO₄, MgSO₄, silica gel, or molecular sieves.

Although some metals/metal oxides are known as oxygen scavengers, generally these compounds are very reactive towards fluoroolefins based on the technical literature. Metals/metal oxides present in known oxygen scavengers, have been used to cleave C—F bonds to form more stable metal fluoride bonds. Cleavage of the C—F bond in fluoroolefins (C═C—F) is quite common. See Ristic-Petrovic, D. et al., Organometallics 2003, 22, 4647-4657, Ahrens, T. et al. Chem. Rev. 2015, 115, 931-972, each incorporated by reference. It was surprising that certain reduced metal oxides of Cu, Ti, V, Mn, Fe, Co, Zn, Ni, and Pd perform the intended function of removing oxygen, but without cleavage of C—F bonds in 2,3,3,3-tetrafluoropropene or other fluoroolefins.

Metal-fluorine bonds (e.g., Cu—F˜431 KJ/mol) are more stable than metal-oxygen (e.g., Cu—O˜343 KJ/mol) bonds, so there is an energetic driving force for reaction of metal oxides (the products of oxygen scavenging) with organofluorine compounds. Thus, it was not expected and unobvious that materials useful for scrubbing oxygen from inert gases (Ar, N₂), hydrocarbons, and regular olefins (ethylene, propylene) could remove oxygen contaminants from 2,3,3,3-tetrafluoropropene without significant degradation of the organofluorine compounds.

Representative additional HFO, HFCO, HFC, HCC, HCFC components, include, but are not limited to, those listed in Table 1 below.

TABLE 1

| | | Chemical |
|---|---|---|
| Name | Chemical name | Formula |
| HCFO-1112 | Z/E-1,2-dichloro-1,2-difluoroethylene | CClF═CClF |
| HCFO-1113 | Chlorotrifluoroethene | CClF═CF₂ |
| HCFO-1122 | 1-chloro-2,2-difluoroethene | CHCl═CF₂ |
| HCFO-1122a | Z/E-I-chloro-1,2-difluoroethene | CHF═CClF |
| HFO-1123 | 1,1,2-trifluoroethylene | CHF═CF₂ |
| HFO-1132 | Z/E-1,1-difluoroethylene | CHF═CHF |
| HFO1132a | 1,1-difluoroethylene | CH₂═CF₂ |
| HCC-1140 | Chloroethene | CH₂═CHCl |
| HFC-1141 | 1-fluoroethane | CHF═CH₂ |
| HFCO-1224yd | Z/E-1-chloro-2,3,3,3- | CF₃CF═CHCl |
| | tetrafluoropropene | |
| HFO-1225zc | 1,1,3,3,3-pentafluoroprop-1-ene | CF₃CH═CF₂ |
| HFO-1225ye | Z/E-1,2,3,3,3-pentafluoropropene | CF₃CF═CHF |
| HCFO-1233zd | Z/E-1-chloro-3,3,3-trifluoropropene | CF₃CH═CHCl |
| HFO-1234yf | 2,3,3,3-tetrafluoropropene | CF₃CF═CH₂ |
| HFO-1234ze | Z/E-1,3,3,3-tetrafluoropropene | CF₃CH═CHF |
| HFO-1234ye | Z/E-1,2,3,3-tetrafluoropropene | CHF₂CF═CHF |
| HFO-1234zc | 1,1,3,3-tetrafluoro-1-propene | CHF₂CH═CF₂ |
| HFO1234yc | 1,1,2,3-tetrafluoro-1-propene | CH₂FCF═CF₂ |
| HFC-1243zf | 3,3,3-trifluoropropene | CF₃CH═CH₂ |
| HFO-1327mz | Z/E-1,1,1,2,4,4,4-heptafluorobut-2- | CF₃CH═CFCF₃ |
| | ene | |
| HFO-1336mzz | Z/E-1,1,1,4,4,4-hexafluoro-2-butene | CF₃CH═CHCF₃ |
| HCFC-12 | Dichlorodifluoromethane | CCl₂F₂ |
| HCFC-22 | Chlorodifluoromethane | CHClF₂ |
| HFC-23 | Trifluoromethane | CHF₃ |
| HFC-32 | Difluoromethane | CH₂F₂ |
| HCC-40 | Chloromethane | CH₃Cl |
| HCFC-114 | 1,2-dichloro-1,1,2,2- | CClF₂CClF₂ |
| | tetrafluoroethane | |
| HCFC-114a | 1,1-Dichlorotetrafluoroethane | CF₃CCl₂F |
| HCFC-123 | 1,1,1,1-trifluoro-2,2-dichloroethane, | CHCl₂CF₃ |
| HFC-125 | Pentafluoroethane | CF₃CHF₂ |
| HFC-134 | 1,1,2,2-tetrafluoroethane | CHF₂CHF₂ |
| HFC-134a | 1,1,1,2-tetrafluoroethane | CH₂FCF₃ |
| HFC-143a | 1,1,1-trifluoroethane | CH₃CF₃ |
| HFC-152a | 1,1-difluoroethane | CHF₂CH₃ |
| HFC-161 | Fluoroethane | CH₃CH₂F |
| HFC-227ea | 1,1,1,2,3,3,3-heptafluoropropane, | CF₃CHFCF₃ |
| HFC-236fa | 1,1,1,3,3,3-hexafluoropropane, | CF₃CH₂CF₃ |
| HFC-245cb | 1,1,1,2,2-pentafluoropropane | CH₃CF₂CH₃ |
| HFC-263fb | 1,1,1-trifluoropropane | CF₃CH₂H₃ |
| HFC-356mff | 1,1,1,4,4,4-hexafluorobutane | CF₃CH₂CH₂CF₃ |
| HFC-365mfc | 1,1,1,3,3-pentafluorobutane | CF₃CH₂CF₂CH₃ |
| HFC-1429mzy | E/Z-1,1,1,3,4,4,5,5,5-nonafluoro-2- | CF₃CH═CFC₂F₅ |
| | pentene | |
| HFC-1429myz | E/Z-1,1,1,2,4,4,5,5,5-nonafluoro-2- | CF₃CF═CHC₂F₅ |
| | pentene | |
| HFC-1438mzz | E/Z-1,1,1,4,4,5,5,5-octafluoro-2- | CF₃CH═CHC₂F₅ |
| | pentene | |
| HFOC-125E | pentafluorodimethyl ether | CHF₂OCF₃ |
| HFOC-134E | bis(difluoromethyl) ether | CHF₂OCHF₂ |
| HFOC-134aE | fluoromethyl trifluoromethyl ether | CH₂FOCF₃ |
| DME | dimethyl ether | CH₃OCH₃ |
| HFOC-143aE | trifluoromethyl methyl ether | CF₃OCH₃ |
| HFOC-227eaE | trifluoromethyl 1,2,2,2- | CF₃OCHFCF₃ |
| | tetrafluoroethyl ether | |
| HFOC-236eaE | trifluoromethyl 1,2,2-trifluoroethyl | CF₃OCHFCHF₂ |
| | ether | |
| HFOC-236eaE | difluoromethyl 1,2,2,2- | CHF₂OCHFCF₃ |
| | tetrafluoroethyl ether | |
| HFOC-236faE | trifluoromethyl 2,2,2-trifluoroethyl | CF₃OCH₂CF₃ |
| | ether | |
| HFOC-245cbE | methyl pentafluoroethyl ether | CH₃OC₂F₅ |
| HFOC-263fb | trifluoromethyl ethyl ether | CF₃OC₂H₅ |

Certain of the compounds of Table 1 exist as different configurational isomers or stereoisomers. In instances where the specific isomer is not designated, the present invention is intended to include all single configurational isomers, single stereoisomers, single geometric or any combination thereof in any ratio. For example, the designation HFO-Z/E-1234ze is meant to include the E-HFO-HFO1234ze (HFO-1234ze (E)) isomer, the Z-HFO-1234ze (HFO-1234ze (Z)) isomer, and mixtures of the E and Z isomers of HFO-1234ze.

One embodiment of the invention disclosed herein involves contacting a fluoroolefin-containing feed which has an oxygen content of between 100 ppm and about 5000 ppm, e.g., HFO-1234yf and/or HFO-1234ze, with a metal oxide at a temperature sufficient, e.g., 20° C. to 30° C., to reduce or remove oxygen without cleavage of the C—F bond of the HFO-1234yf and recover the substantially oxygen-free HFO-1234yf and/or HFO-Z/E-1234ze.

The fluoroolefin-containing feeds processed in the present invention include one or more hydrofluorocarbon compounds compliant with AHRI standards, where the total amount of air and other non-condensable impurities could facilitate oligomerization/polymerization of a fluoroolefin component such as HFO-1234yf.

In another embodiment of the invention disclosed herein, the substantially oxygen-free HFO-1234yf and/or HFO-Z/E-1234ze is packaged under pressure in a storage vessel.

In another embodiment of the invention disclosed herein, the substantially oxygen-free HFO-1234yf and/or HFO-Z/E-1234ze is blended with at least one of HFO-1243zf, Z-HFO-1336mzz, E-HFO-1336mzz, HFO-Z/E-1327mz, HCFO-1122, HCFO-Z/E-1122a, HFO-1123, HCFO-Z/E-1233zd, HCFO-Z/E-1224 yd, HFO-1132a, and HFO-Z/E-1132, CFO-Z/E-1112, E-HFO-1225ye, Z-HFO-1225ye, HFO-1234zc, HFO-Z/E-1234ye, HFO-1234yc, HFO-1225zc, and HFC-152a, in which oxygen has been reduced or removed.

The present invention also involves removing stabilizers from fluoroolefin compositions to produce high purity HFO-1234yf and/or HFO-Z/E-1234ze compositions, along with any additional HFO, HFCO, HFC, HCC, or HCFC components. U.S. Patent Publication Nos. US2021/0108119 and US2021/0040368, each disclosure of which is hereby incorporated herein by reference in its entirety, discloses that an oligomer or polymer inhibitor is added to HFO-1234yf and/or HFO-Z/E-1234ze compositions and blends. While these stabilizer compounds effectively react with oxygen and/or function as chain transfer agents to terminate the polymer chain propagation of e.g., HFO-1234yf, their presence also reduces the purity of the refrigerant going to a refrigerant system. In order to return the stabilized composition to the desired high purity, it is necessary to remove the stabilizer. Contacting the stabilized HFO-1234yf compositions with silica or mineral oil removes the stabilizer, as well any moisture that may be present and produces high purity HFO-1234yf and/or HFO-1234ze containing compositions.

In certain embodiments disclosed herein, the inhibiting stabilizer that is added or removed includes, but is not limited to, d-Limonene and α-terpinene having the following structures:

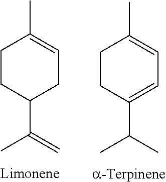

In another embodiment disclosed herein, the inhibiting stabilizer includes, but is not limited to, a-tocopherol, butylated hydroxytoluene, 4-methoxyphenol, and benzene-1,4-diol.

In another embodiment disclosed herein, the inhibiting stabilizer includes at least two inhibiting agents, e.g., pairs, including, but not limited, to limonenes, pinenes, terpinenes, ethane, propane, cyclopropane, propylene, butane, butene, isobutane, and isobutene.

In another embodiment disclosed herein, the inhibiting stabilizer includes, but is not limited to a limonene and propane, or a pinene and propane.

The additional one or more HFO, HCFO, HFC, HCC, or HCFC components include, but are not limited to HFO-1243zf, HFO-Z-1336mzz, HFO-E-1336mzz, HFO-Z/E-1327mz, HCFO-1122, HCFO-Z/E-1122a, HFO-1123, HCFO-Z/E-1233zd, HCFO-Z/E-1224 yd, HFO-Z/E-1132, HFO-1132a, CFO-Z/E-1112, E-HFO-1225ye, Z-HFO-1225ye, HFO-1234zc, HFO-Z/E-1234ye, HFO-1234yc, HFO-1225zc, and HFC-152a (see Table 1).

In one embodiment disclosed herein, HFO-1234yf alone or including at least one of HFC-32, HFC-125, HFC-134a, and carbon dioxide includes one of d-Limonene and α-terpinene.

In another embodiment disclosed herein, HFO-1234yf and HFO-Z/E-1234ze include one of d-Limonene and α-terpinene, and optionally at least one of the following additional components: HFO-1243zf, HFO-Z-1336mzz, HFO-E-1336mzz, HFO-Z/E-1327mz, HFO-1122, HFO-Z/E-1122a, HFO-1123, HFO-Z/E-1233zd, HFO-1224 yd, HFO-Z/E-1132, HFO-1132a, HCFO-1112, HFO-E-1225ye, HFO-Z-1225ye, HFO-1234zc, HFO-Z/E-1234ye, HFO-1234yc, HFO-1225zc, and HFC 152a.

### Definitions

As used herein a refrigerant is a compound or mixture of compounds that functions as a heat transfer fluid in a cycle wherein the fluid undergoes a phase change from a liquid to a gas and back.

As used herein, a refrigeration circuit may be part of a mobile or stationary heat transfer system fixed in place during operation. A stationary heat transfer system may be located within, attached to, or otherwise associated with buildings or any stand-alone device located outdoors, such as a soft drink vending machine. These stationary applications may include, but not limited to stationary air conditioning units and heat pumps; chillers; high temperature heat pumps; and residential, commercial, or industrial air conditioning systems (including residential heat pumps). In stationary refrigeration applications, the disclosed stabilized or purified compositions or blends may be useful in various items of equipment, including commercial, industrial, or residential refrigerators and freezers; ice machines; self-contained coolers and freezers; flooded evaporator chillers; direct expansion chillers; walk-in and reach-in coolers and freezers; and combination systems. In some embodiments, the disclosed compositions may be used in supermarket refrigeration systems. Additionally, stationary applications may utilize a secondary loop system, where a secondary fluid is cooled by a primary refrigerant, and the secondary fluid is then pumped to a remote location in order to provide a cooling effect at that remote location.

A heat transfer medium, such as a refrigerant (also referred to herein as a heat transfer fluid, a heat transfer composition or a heat transfer fluid composition or refrigerant composition) is a working fluid used to carry heat from a heat source to a heat sink.

As used herein, the term “lubricant” means any material added to a composition or a compressor (and in contact with any heat transfer composition in use within any heat transfer system) that provides lubrication to the compressor to aid in preventing parts from seizing.

As used herein, compatibilizers are compounds which improve solubility of the working fluids of the disclosed compositions in heat transfer system lubricants. In some embodiments, the compatibilizers improve oil return to the compressor. In some embodiments, the composition is used with a system lubricant to reduce oil-rich phase viscosity.

As used herein, the phrase “substantially inhibitor-free” means an inhibitor content of about 10 ppm or less including 0 ppm, preferably about 5 ppm or less including 0 ppm, and all values and ranges therebetween.

As used herein, the phrase “moisture-free” means a moisture content of about 10 ppm or less including 0 ppm, preferably about 5 ppm or less including 0 ppm, and all values and ranges therebetween.

As used herein, the phrase “oxygen-free” means an oxygen content of about 10 ppm or less including 0 ppm, preferably about 5 ppm or less including 0 ppm, and all values and ranges therebetween.

By “inhibitor” it is meant to refer to at least one compound in accordance with the present invention that reduces, if not eliminates, conversion of hydrofluoroolefins into oligomers or polymers. While oligomerization or homopolymerization reactions may be accelerated by relatively high temperatures, such reactions may also occur under ambient conditions depending upon the concentration and type of initiator (e.g., contaminant). The inhibitor can function as a radical inhibitor or a chain transfer reagent without affecting the refrigeration performance or compatibility of the composition with refrigerant oil(s) and equipment (e.g., resins used in seals). The stabilized compositions may be useful in cooling/heating systems and as replacements for existing refrigerants with higher global warming potential.

The term fluoroolefin, as used herein, describes unsaturated compounds which comprise carbon atoms, fluorine atoms, and optionally hydrogen atoms. The term chlorofluoroolefin, as used herein, describes unsaturated compounds which comprise carbon atoms, chlorine atoms, fluorine atoms, and optionally hydrogen atoms. In one embodiment, the fluoroolefins used in the compositions of the present invention comprise compounds with 3-12 carbon atoms. In another embodiment the fluoroolefins comprise compounds with 3 to 10 carbon atoms, and in yet another embodiment the fluoroolefins comprise compounds with 3 to 7 carbon atoms.

As described herein, the phrase “effective amount” refers to an amount of inhibitor of the present invention which, when added to a composition comprising at least one fluoroolefin, prevents oligomerization or polymerization of the fluoroolefin with no reduction in performance, for example, when in use in a cooling apparatus as compared to the composition without an inhibitor. For cooling apparatus, such effective amounts of inhibitor may be determined by way of testing under the conditions of standard test ASHRAE 97-2007 (RA 2017).

In one embodiment of the invention, HFO-1234yf from a storage vessel, without any inhibitor, is first analyzed using known techniques, such as GC-MS, to determine the compositional make-up, and optionally the oxygen content. In one embodiment, the oxygen content is generally between about 100 ppm and about 5000 ppm or more. Suitable techniques to analyze the oxygen content include, but are not limited to, infrared sensors, UV sensors, NIR sensors, ion mobility or plasma chromatographs, gas chromatography, refractometry, mass spectroscopy, high temperature thick film sensors, thin film field effect sensors, pellistor sensors, Taguchi sensors and quartz microbalance sensors as disclosed in U.S. Patent Application Publication No. 2008/0069177, the entire disclosure of which is incorporated herein by reference, in particular column 3, line 42 through column 8, line 15. The HFO-1234yf then contacts a reduced metal oxide under ambient conditions to remove or reduce any oxygen present and is again tested as described above. The substantially oxygen-free HFO-1234yf, e.g., containing less than about 10 ppm, less than 5 ppm, less than 4 ppm, less than 3 ppm, or preferably less than 2 ppm is repackaged in storage vessels as neat HFO-1234yf or blended with other components which have been similarly treated to reduce the oxygen content to less than about 10 ppm, less than 5 ppm and all values therebetween.

In another embodiment of the invention, an HFO-1234yf composition which contains an oligomerization or polymerization inhibitor, including but not limited to limonenes, pinenes and terpinenes, and their oxidation products, is optionally analyzed using known techniques to determine the compositional make-up, and identify the inhibitor, and optionally determine the oxygen content. Suitable oxygen analyzers include, but are not limited to infrared sensors, UV sensors, NIR sensors, ion mobility or plasma chromatographs, gas chromatography, refractometry, mass spectroscopy, high temperature thick film sensors, thin film field effect sensors, pellistor sensors, Taguchi sensors and quartz microbalance sensors. Thereafter, the composition contacts a bed of silica gel which removes the inhibitor and any moisture that may be present. The composition may be tested again to verify that the inhibitor and any moisture has been removed, and if acceptable used as a source of pure HFO-1234yf which may then be charged to refrigeration equipment. Optionally, once the inhibitor and moisture have been removed, the composition may be further contacted with a reduced metal oxide to reduce or remove any oxygen that may be present, and then optionally analyzed again to determine the oxygen content.

In certain embodiments, the HFO-1234yf and/or HFO-1234ze supply includes, but is not limited to, feed streams or storage vessels, e.g., container, tanks, or cannisters, and the supply of other HFO, HCFO, HFC, HCC, HCFC components, may similarly include, but is not limited to, feed streams or storage vessels of any design suitable for storing HFOs, HCFOs, HFCs, HCCs, and HCFCs.

In certain embodiments of the present invention, substantially oxygen-free HFO-1234yf and HFC-32 are blended. Difluoromethane (HFC-32 or R-32) is commercially available or may be made by methods known in the art, such as by dechlorofluorination of methylene chloride. In either case, HFC-32 will be processed to have an oxygen content of less than about 10 ppm, preferably less than about 5 ppm.

In one embodiment, the HFC-32 component of the inventive composition comprises HFC-32 having a purity of greater than 99 wt. %, greater than 99.5 wt. %, and in some cases greater than 99.5 to 99.98 weight percent, and after contacting a reduced metal oxide, the oxygen content of the HFC-32 is less than 10 ppm, less than 5 ppm and all values therebetween.

In another particular embodiment, the HFC-32 component comprises greater than 99.99 wt. % HFC-32, and after contacting a metal oxide, the oxygen content of the HFC-32 will be 10 ppm or less, 5 ppm or less, and all values therebetween. In one embodiment, the HFC-32 component further comprises at least one additional compound selected from HFC-23 (trifluoromethane), HCFC-31 (chlorofluoromethane), HFC-41 (fluoromethane), HFC-143a (1,1,1-trifluoroethane), HCFC-22 (chlorodifluoromethane), CFC-12 (dichlorodifluoromethane), HCC-40 (chloromethane), and HFC-134a (1,1,1,2-tetrafluoroethane), and each is treated to have an oxygen content of less than about 10 ppm, less than about 5 ppm and all values therebetween.

In particular embodiments, the inhibitor-containing compositions may contain:
about 31 wt. % HFO-1234yf and about 69 wt. % HFC-32; or
about 65 wt. % HFO-1234yf and about 35 wt. % HFC-32; or
about 78.5 wt. % HFO-1234yf and about 21.5 wt. % HFC-32.

In another particular embodiment, the inhibitor-containing composition may contain:
from about 20 to about 40 weight percent HFO-1234yf; or
from about 28 to about 32 weight percent HFO-1234yf; or
from about 30 to about 32 weight percent HFO-1234yf; or
from about 62 to about 65 weight percent HFO-1234yf; or
from about 76 to about 80 weight percent HFO-1234yf.

In one embodiment, inhibitor and/or moisture-free HFO-1234yf includes HFC-134a or HFC-32 in particular weight ratios. The amount of the HFC-134a can range from about 25 to about 75 wt. %, about 30 to about 60 wt. %, and in some cases about 30 to about 50 wt. %. HFO-1234yf blends or compositions include,
a. about 14 weight percent HFC-134a; or
b. about 25.7 weight percent HFC-134a; or
c. about 25 weight percent to 45 weight percent HFC-134a; or
d. about 25 weight percent to 44 weight percent HFC-134a; or
e. about 30 weight percent HFC-125, about 14 weight percent HFC-134a; or
f. about 36 weight percent HFC-32, and about 6 weight percent CO₂; or
g. about 28 to about 32 weight percent HFC-32; or
h. about 68 weight percent to 72 weight percent HFC-32.

In some embodiments, optional other components (also referred to herein as additives) which are included in the fluoroolefin feed, component, composition, or blend disclosed herein may comprise one or more components selected from lubricants, dyes (including UV dyes), solubilizing agents, compatibilizers, stabilizers, tracers, perfluoropolyethers, anti-wear agents, extreme pressure agents, corrosion and oxidation inhibitors, metal surface energy reducers, metal surface deactivators, free radical scavengers, foam control agents, viscosity index improvers, pour point depressants, detergents, viscosity adjusters, and mixtures thereof. Indeed, many of these optional other components fit into one or more of these categories and may have qualities that lend themselves to achieve one or more performance characteristics.

In some embodiments, one or more additive(s) is (are) present in small amounts relative to the overall composition. In some embodiments, the amount of additive(s) concentration in the disclosed compositions is from less than about 0.1 weight percent to as much as about 10 weight percent, preferably about 5 weight percent, of the total composition. In some embodiments of the present invention, the additives are present in the disclosed compositions in an amount between about 0.1 weight percent to about 3.5 weight percent of the total composition. The additive component(s) selected for the disclosed composition is selected on the basis of the utility and/or individual equipment components or the system requirements.

In some embodiments, the lubricant is a mineral oil lubricant. In some embodiments, the mineral oil lubricant is selected from paraffins (including straight carbon chain saturated hydrocarbons, branched carbon chain saturated hydrocarbons, and mixtures thereof), naphthenes (including saturated cyclic and ring structures), aromatics (those with unsaturated hydrocarbons containing one or more ring, wherein one or more ring is characterized by alternating carbon-carbon double bonds) and non-hydrocarbons (those molecules containing atoms such as sulfur, nitrogen, oxygen and mixtures thereof), and mixtures and combinations of thereof.

Some embodiments may contain one or more synthetic lubricant. In some embodiments, the synthetic lubricant is selected from alkyl substituted aromatics (such as benzene or naphthalene substituted with linear, branched, or mixtures of linear and branched alkyl groups, often generically referred to as alkylbenzenes), synthetic paraffins and naphthenes, poly (alpha olefins), polyglycols (including polyalkylene glycols), dibasic acid esters, polyesters, neopentyl esters, polyvinyl ethers (PVEs), silicones, silicate esters, fluorinated compounds, phosphate esters, polycarbonates and mixtures thereof, meaning mixtures of the any of the lubricants disclosed in this paragraph.

The lubricants as disclosed herein may be commercially available lubricants. For instance, the lubricant may be paraffinic mineral oil, sold by BVA Oils as BVM 100 N, naphthenic mineral oils sold by Crompton Co. under the trademarks Suniso® 1GS, Suniso® 3GS and Suniso® 5GS, naphthenic mineral oil sold by Pennzoil under the trademark Sontex® 372LT, naphthenic mineral oil sold by Calumet Lubricants under the trademark Calumet® RO-30, linear alkylbenzenes sold by Shrieve Chemicals under the trademarks Zerol® 75, Zerol® 150 and Zerol® 500 and branched alkylbenzene sold by Nippon Oil as HAB 22, polyol esters (POEs) sold under the trademark Castrol® 100 by Castrol, United Kingdom, polyalkylene glycols (PAGs) such as RL-488A from Dow (Dow Chemical, Midland, Mich.), and mixtures thereof, meaning mixtures of any of the lubricants disclosed in this paragraph.

The lubricants used with the present invention may be designed for use with hydrofluorocarbon refrigerants and may be miscible with compositions as disclosed herein under compression refrigeration and air-conditioning apparatus' operating conditions. In some embodiments, the lubricants are selected by considering a given compressor's requirements and the environment to which the lubricant will be exposed.

In the compositions of the present invention including a lubricant, the lubricant is present in an amount of less than 5.0 weight percent to the total composition. In other embodiments, the amount of lubricant is between about 0.1 and 3.5 weight percent of the total composition.

In one embodiment of the invention, an HFO-1234yf containing refrigerant from a storage supply or an existing system is treated to increase the purity of the refrigerant by removing any oligomer inhibitor and at least one of moisture and oxygen and then charged to refrigerant equipment, including but not limited to commercial, industrial, or residential refrigerators and freezers; ice machines; self-contained coolers and freezers; flooded evaporator chillers; direct expansion chillers; walk-in and reach-in coolers and freezers; and combination systems. In some embodiments, the disclosed compositions may be used in supermarket refrigeration systems. Additionally, stationary applications may utilize a secondary loop system, where a secondary fluid is cooled by a primary refrigerant, and the secondary fluid is then pumped to a remote location in order to provide a cooling effect at that remote location.

The containers for storing the high purity HFO-1234yf and/or HFO-E/Z-1234ze, or blends with at least one additional HFO, HCFO, HFC, HCC, HCFC, and carbon dioxide (CO₂) can be constructed of any suitable material and design that is capable of sealing the compositions therein while maintaining gaseous and liquids phases. Examples of suitable containers comprise pressure resistant containers such as a tank, a filling cylinder, and a secondary filing cylinder. The container can be constructed from any suitable material such as carbon steel, manganese steel, chromium-molybdenum steel, among other low-alloy steels, stainless steel, and in some cases an aluminum alloy. The container can include a pierceable top or valves suitable for dispensing flammable substances.

In certain embodiments of the invention, (1) refrigerant equipment comprises at least one refrigerant circuit filled with substantially oxygen-free HFO-1234yf and/or HFO-Z/E-1234ze, for example HFO-1234yf and HFO-Z/E-1234ze containing less than 10 ppm to 0 ppm oxygen, or less than 5 ppm to 0 ppm oxygen, optionally including at least one additional component comprising a substantially oxygen-free component selected from HFC-125, HFC-134a, HFC-134, HFC 152a, HFC-23, HCFO-Z/E-1233zd, and HFO-Z/E-1336mzz; (2) refrigerant equipment comprises a refrigerant circuit filled with between about 0.1 and about 99.9 wt. % HFO-1234yf and between about 0.1 and about 99.9 wt. % HFO-Z/E-1234ze which contains less than 5 ppm to 0 ppm of an oligomer inhibitor, optionally including at least one additional component comprising HFC-125, HFC-134a, HFC-134, HFC 152a, HFC-23, HCFO-Z/E-1233zd, and HFO-Z/E-1336mzz; and (3) refrigerant equipment comprises at least one refrigerant circuit filled with between about 0.1 and about 99.9 wt. % HFO-1234yf and between about 0.1 and about 99.9 wt. % HFO-Z/E-1234ze, optionally including at least one additional component comprising HFC-125, HFC-134a, HFC-134, HFC 152a, HFC-23, HFC-227ea, HCFO-Z/E-1233zd, HFO-Z/E-1132, HFO-1132a, and HFO-Z/E-1336mzz, and substantially inhibitor- and/or moisture- and/or oxygen-free.

In certain embodiments of the invention, a storage vessel is filled with (1) pressurized substantially oxygen-free HFO-1234yf and/or HFO-Z/E-1234ze, preferably HFO-1234yf and HFO-Z/E-1234ze containing less than 10 ppm, less than 5 ppm to 0 ppm oxygen, and all values therebetween, optionally including at least one additional substantially oxygen-free components selected from HFC-125, HFC-134a, HFC-134, HFC 152a, HFC-23, HFC-227ea, HCFO-Z/E-1233zd, and HFO-Z/E-1336mzz; (2) between about 0.1 and about 99.9% HFO-1234yf and between about 0.1 and about 99.9% HFO-Z/E-1234ze with less than 5 ppm to 0 ppm of an oligomer inhibitor, optionally including at least one additional component comprising HFC-125, HFC-134a, HFC-134, HFC 152a, HFC-23, HCFO-Z/E-1233zd, and HFO-Z/E-1336mzz; and (3) between about 0.1 and about 99.9% HFO-1234yf and about 0.1 and about 99.9% HFO-Z/E-1234ze, optionally including at least one additional component comprising HFC-125, HFC-134a, HFC-134, HFC 152a, HFC-23, HFO-Z/E-1132, HFO-1132a, HCFO-Z/E-1233zd, and HFO-Z/E-1336mzz, and substantially inhibitor- and/or moisture- and/or oxygen-free meaning the composition contains less than about 10 ppm, preferably less than about 5 ppm of an inhibitor and/or moisture and/or oxygen, respectively.

In other embodiments of the present invention, the substantially inhibitor- and/or moisture- and/or oxygen-free fluoroolefin compositions are employed for cooling and or heating in heat transfer systems including, but not limited to, air conditioners, freezers, refrigerators, heat pumps, water chillers, flooded evaporator chillers, direct expansion chillers, walk-in coolers, mobile refrigerators, mobile air conditioning units, dehumidifiers, and combinations thereof.

### EXAMPLES

### Examples 1-4

An Inconel Tube® (0.5-inch OD, 15-inch length, 0.34 in wall thickness) is filled with 8 cc of CuO (40% by weight) on Al₂O₃. The catalyst is reduced by 50 sccm H₂/N2 (50% H₂) in a Lindberg furnace to 175° C. for 8 hours. After the reactor is cooled to room temperature, CF₃CF═CH₂ (HFO-1234yf) containing different concentrations of air is fed at 10-20 sccm (standard cubic centimeters per minute). Over the course of the run, the air concentration is measured by GC and shows O₂ concentration is below 5 ppm.

| | | O₂ concentration |
|---|---|---|
| Example | Air concentration (ppm) | after treatment (ppm) |
| 1 | 1,000 | <5 |
| 2 | 2,000 | <5 |
| 3 | 3,000 | <5 |
| 4 | 6,000 | <5 |

### Examples 5-10

30 g HFO-1234yf containing various concentration of d-limonene is passed 20 g silica gel (200 mesh) or 50 mL mineral oil. GC shows the residual inhibitor content is less than 5 ppm.

| | | d-limonene | d-limonene |
|---|---|---|---|
| | | Concentration | concentration after |
| Example | Absorbent | before treatment | treatment (ppm |
| 5 | silica gel | 100 | <5 |
| 6 | silica gel | 200 | <5 |
| 7 | silica gel | 400 | <5 |
| 8 | mineral oil | 100 | <5 |
| 9 | mineral oil | 200 | <5 |
| 10 | mineral oil | 400 | <5 |

### Other Embodiments

Process embodiment 1 of the present invention comprises:
a) contacting a fluoroolefin feed containing at least HFO-1234yf and an oxygen content of up to about 5000 ppm with a reduced metal oxide at a temperature sufficient to reduce or remove oxygen from the fluoroolefin feed without cleavage of a C—F bond of at least one fluoroolefin; and
b) recovering a substantially oxygen-free fluoroolefin product, wherein the metal oxide comprises at least one of Cu, Ti, V, Mn, Fe, Co, Zn, Ni and Pd as the metal and the temperature is room temperature.

Process embodiment 2 of the present invention comprises:
a) contacting a fluoroolefin feed and an oligomer/polymer inhibitor with one of silica gel and mineral oil at a temperature sufficient to remove the inhibitor; and
b) recovering a substantially oligomer/polymer inhibitor-free fluoroolefin product.

In process embodiment 2, the fluoroolefin feed has an inhibitor content up to 5 weight percent and silica gel is used to remove the inhibitor to less than 5 ppm.

Process embodiment 2 further comprises,
c) contacting the inhibitor-free fluoroolefin product of step b) with a reduced metal oxide at a temperature sufficient to reduce or remove oxygen without cleavage of a C—F bond of the fluoroolefin; and
d) recovering a substantially oxygen-free and inhibitor-free fluoroolefin feed.

The process embodiments 1 and 2, wherein the temperature is between 20° C. and 30° C.; or the fluoroolefin feed has an oxygen content of between 100 ppm and about 5000 ppm; or the fluoroolefin product has an oxygen content of at most about 5 ppm oxygen.

The process embodiments 1 and 2, wherein the temperature is between 20° C. and 30° C., the fluoroolefin feed has an oxygen content of between 100 ppm and about 5000 ppm, and the fluoroolefin product has an oxygen content of at most about 5 ppm oxygen.

The process embodiment 3, wherein the feed of process embodiment 2 comprises:
i. HFO-1234yf; or
ii. HFO-Z/E-1234ze; or
iii. a mixture of HFO-1234yf and HFO-Z/E-1234ze.

The process embodiment 1, or 2 or 3, wherein at least one additional HFO, HCFO, HFC, HCFC, and carbon dioxide (CO₂) component is blended with the substantially oxygen-free product.

The process embodiment 2 or 3, wherein the oligomer/polymer inhibitor includes at least one oligomer/polymer inhibitor comprising at least one of ethane, propane, cyclopropane, propylene, butane, butene, isobutane, isobutene, meta-, ortho-, or para-xylene, alpha (α)-methyl styrene, 2-methyl-alpha-methylstyrene (α, 2-dimethylstyrene), 3-methyl-alpha-methylstyrene (α, 3-dimethylstyrene) and 4-methyl-alpha-methylstyrene (α, 4-dimethylstyrene) and mixtures of two or more of said inhibitors.

The process embodiment 1, or 2 or 3, wherein at least one additional component suitable for heat transfer is blended with the substantially oxygen-free product and is selected from at least one of HFC-125, HFC-134a, HFC-134, HFC-227ea, HCC-23, CHFO-1233zd (E/Z), and HFO-ZE-1336mzz.

The process embodiment 1, or 2 or 3, wherein at least one additional substantially oxygen-free component is blended with the product.

The process embodiment 1, 2 or 3, further comprising determining the oxygen content of the at least one fluoroolefin feed.

The process embodiment 1, 2 or 3, wherein the feed includes an additional component comprising:
HFO-Z/E-1225ye; or HFO-Z/E-1225ye and HFC-32; or HFO-Z/E-1225ye and HFC-134a; or HFC 152a, or HFO-Z/E-1225ye, HFC-134a, and HFC-32; or HFO-Z/E-1225ye and HFC-125; or at least one of HCC-40, HCFC-22, CFC-115, HCFC-124, HCFC-1122, and CFC-1113; or FC-125, HFC-134a, HFC-134, HFC-23, HCFO-Z/E-1233zd, and HFO-Z/E-1336mzz.

Process embodiment 4 of the present invention provides a process comprising: passing a fluoroolefin composition through a column containing a reduced metal oxide wherein the reduced metal oxide comprises at least one of Cu, Ti, V, Mn, Fe, Co, Zn, Ni and Pd as the metal, said fluoroolefin composition comprising one of: HFO-1234yf; HFO-Z/E-1234ze; and a mixture of HFO-1234yf and HFO-Z/E-1234ze; operating the column at ambient temperature to remove and/or reduce oxygen in the fluoroolefin composition to at most 10 ppm, preferably at most 5 ppm, without cleavage of the C—F bond of the fluoroolefin; and, recovering a fluoroolefin containing less than 10 ppm oxygen, preferably less than 5 ppm oxygen.

Process embodiment 4 of the present invention uses a fluoroolefin feed which also contains one or more of HFC-125, HFC-134a, HFC-134, HFC 152a, HFC-23, HCFO-Z/E-1233zd, and HFO-Z/E-1336mzz, optionally treated with a reduced metal oxide containing column operated at ambient temperature to provide a substantially oxygen-free HFC-125, HFC-134a, HFC-134, HFC-23, HCFO-Z/E-1233zd, and HFO-Z/E-1336mzz component; and optionally blended with i), or ii) or iii).

Pressurized supply embodiment 1 of the present application provides a pressure-resistant-container filled with a refrigerant comprising a pierceable top or valve adapted to dispense a refrigerant which comprises at least one of substantially oxygen-free HFO-1234yf; substantially oxygen-free HFO-Z/E-1234ze; and a mixture of HFO-1234yf and HFO-Z/E-1234ze containing less than 10 ppm to 0 ppm oxygen, preferably less than 5 ppm to 0 ppm oxygen.

Pressurized supply embodiment 2 of the present application includes the refrigerant of pressurized supply embodiment 1 and an additional refrigerant component which is substantially oxygen-free and comprises HFC-125, or HFC-134a, or HFC-134, or HFC-23, or HCFO-Z/E-1233zd, or HFO-Z/E-1336mzz, each containing less than 10 ppm to 0 ppm oxygen, preferably less than 5 ppm to 0 ppm oxygen.

Refrigerant equipment embodiment 1 of the present invention comprises at least one refrigerant circuit pressurized with 0.1 to about 99.9 wt. % HFO-1234yf and between about 0.1 and about 99.9 wt. % HFO-Z/E-1234ze which contains less than 5 ppm to 0 ppm of an oligomer inhibitor, optionally including at least one additional component selected from HFC-125, HFC-134a, HFC-134, HFC-23, E-HCFO-1233zd, Z-HCFO-1233zd, E-HFO-1336mzz and Z-HFO-1336mzz.

Refrigerant equipment embodiment 2 of the present invention comprises at least one refrigerant circuit pressurized with a composition comprising between about 0.1 and about 99.9 wt. % HFO-Z/E-1234yf and about 0.1 and about 99.9 wt. % HFO-Z/E-1234ze, optionally including at least one additional component selected from HFC-125, HFC-134a, HFC-134, HFC 152a, HFC-23, HCFO-Z/E-1233zd, and HFO-Z/E-1336mzz, and the composition being substantially inhibitor- and/or moisture- and/or oxygen-free.

Process embodiment 5 of present invention provides a method comprising: passing a fluoroolefin composition containing at least HFO-1234yf and optionally an oligomer/polymer inhibitor through one of:
a) silica gel or mineral oil at a temperature sufficient to remove any inhibitor; and
b) a column containing a reduced metal oxide wherein the metal oxide comprises at least one of Cu, Ti, V, Mn, Fe, Co, Zn, Ni and Pd as the metal and operating the column at ambient temperature to remove and/or reduce oxygen in the fluoroolefin composition to at most 10 ppm, preferably at most 5 ppm, without cleavage of the C—F bond of the fluoroolefin, and
c) recovering an HFO-1234yf-containing fluoroolefin containing at least one of less than 10 ppm oxygen, preferably less than 5 ppm oxygen, and less than 5 ppm oligomer inhibitor.

Packaging embodiment 1 of the present invention provides packaging the product of process embodiments 1, 2 3, 4 or 5.

The process embodiments 1, 2, 3, 4 or 5 wherein the feed comprises unreclaimed refrigerant, storage refrigerant, fresh refrigerant, or recycled refrigerant, each of which can have an oxygen content of up to about 5000 ppm.

Process embodiment 6 of the present invention comprises charging refrigerant equipment with substantially inhibitor- and/or moisture- and/or oxygen-free fluoroolefins of Process embodiments 1, 2, 3, 4 or 5.

Process embodiment 7 of the present invention comprises cooling or heating using heat transfer equipment with substantially inhibitor- and/or moisture- and/or oxygen-free fluoroolefins of Process embodiments 1, 2, 3, 4 or 5.

Heating and cooling embodiments of the invention include using the inhibitor- and/or moisture- and/or oxygen-free fluoroolefin products of embodiments 1, 2, 3, 4 or 5 in air conditioners, freezers, refrigerators, heat pumps, water chillers, flooded evaporator chillers, direct expansion chillers, walk-in coolers, mobile refrigerators, mobile air conditioning units, dehumidifiers, and combinations thereof.

Although certain aspects, embodiments and principals have been described above, it is understood that this description is made only way of example and not as limitation of the scope of the invention or appended claims. The foregoing various aspects, embodiments and principals can be used alone and in combinations with each other.

What is claimed is:

1. A process comprising:
a) contacting a fluoroolefin feed containing oxygen and comprising at least one tetrafluoropropene compound with a reduced metal oxide at a temperature sufficient to reduce or remove oxygen from the fluoroolefin feed without cleavage of a C—F bond of the at least one tetrafluoropropene; and
b) recovering a substantially oxygen-free fluoroolefin product which has an oxygen content of at most about 10 ppm oxygen.

2. The process according to claim 1, wherein the reduced metal oxide comprises at least one metal selected from the group consisting of Cu, Ti, V, Mn, Fe, Co, Zn, Ni and Pd.

3. (canceled)

4. A process comprising:
a) contacting an oxygen-containing fluoroolefin feed comprising at least one tetrafluoropropene compound and an oligomer/polymer inhibitor with one of silica gel and mineral oil at a temperature sufficient to reduce or remove the oligomer/polymer inhibitor; and
b) recovering a substantially oligomer/polymer inhibitor-free fluoroolefin product which has an oligomer/polymer inhibitor content of less than 10 ppm.

5. The process according to claim 3, wherein the fluoroolefin feed has an inhibitor content of greater than 10 ppm and up to 5 weight percent and silica gel is used to reduce or remove the inhibitor content.

6. (canceled)

7. The process according to claim 4, further comprising:
c) contacting the fluoroolefin product of step b) with a reduced metal oxide at a temperature sufficient to reduce or remove oxygen without cleavage of a C—F bond of the at least one tetrafluoropropene; and
d) recovering a substantially oxygen-free and inhibitor-fee fluoroolefin product which has an oxygen content of less than about 10 ppm.

8. The process according to claim 1, wherein the temperature is between 20° C. and 30° C.

9. The process according to claim 1, wherein the fluoroolefin feed has an oxygen content of between 100 ppm and about 5000 ppm.

10. The process according to claim 1, wherein the fluoroolefin product has an oxygen content of at most about 5 ppm oxygen.

11. The process according to claim 1, wherein the at least one tetrafluoropropene comprises one of:
i. HFO-1234yf;
ii. HFO-Z/E-1234ze; and
iii. a mixture of HFO-1234yf and HFO-Z/E-1234ze.

12. (canceled)

13. The process according to claim 4, wherein the oligomer/polymer inhibitor includes at least one oligomer/polymer inhibitor comprising at least one material selected from the group consisting of ethane, propane, cyclopropane, propylene, butane, butene, isobutane, isobutene, meta-xylene, ortho-xylene, para-xylene, alpha (α)-methyl styrene, 2-methyl-alpha-methylstyrene (a, 2-dimethylstyrene), 3-methyl-alpha-methylstyrene (a, 3-dimethylstyrene) and 4-methyl-alpha-methylstyrene (a, 4-dimethylstyrene) and mixtures of two or more thereof.

14. The process according to claim 11, wherein at least one additional component is blended with the fluoroolefin feed comprising at least HFO-1234yf, wherein at least one additional component is a component suitable for heat transfer and is selected from the group consisting of HFC-125, HFC-134a, HFC-134, HFC 152a, HCC-23, CHFO-1233zd (E/Z), and HFO-Z/E-1336mzz.

15. The process according to claim 14, further comprising:
c) contacting the at least one additional component with a reduced metal oxide to reduce oxygen concentration to below 5 ppm to produce a substantially oxygen-free additional component prior to blending; and
d) recovering the substantially oxygen-free additional component.

16. (canceled)

17. The process according to claim 1 further comprising: e) determining the oxygen content of the at least one fluoroolefin feed and additional component, and a mixture of the substantially oxygen-free fluoroolefin product and additional component.

18. The process according to claim 17, wherein the oxygen content is determined using an apparatus selected from the group consisting of infrared sensors, UV sensors, NIR sensors, ion mobility or plasma chromatographs, gas chromatography, refractometry, mass spectroscopy, high temperature thick film sensors, thin film field effect sensors, and pellistor sensors.

19. (canceled)

20. (canceled)

21. A method, comprising:
a) passing a fluoroolefin composition through a column containing a reduced metal oxide wherein the reduced metal oxide comprises at least one of Cu, Ti, V, Mn, Fe, Co, Zn, Ni and Pd as the metal, said fluoroolefin composition comprising one of:
i. HFO-1234yf;
i. HFO-Z/E-1234ze; and
iii. a mixture of HFO-1234yf and HFO-Z/E-1234ze;
b) operating the column at ambient temperature to remove and/or reduce oxygen in the fluoroolefin composition to at most 10 ppm, preferably at most 5 ppm, without cleavage of the C—F bond of the fluoroolefin; and
c) recovering a fluoroolefin containing less than 10 ppm, preferably less than 5 ppm, oxygen.

22-24. (canceled)

25. The process according to claim 10, which further comprises:
a) treating least one additional component comprising one of HFC-125, HFC-134a, HFC-134, HFC 152a HFC-23, HCFO-Z/E-1233zd, and HFO-Z/E-1336mzz with a reduced metal oxide-containing column operated at ambient temperature to provide a substantially oxygen-free HFC-125, HFC-134a, HFC-134, HFC-23, HFC 152a, HCFO-Z/E-1233zd, and HFO-Z/E-1336mzz component which has an oxygen content of about 10 ppm or less; and
b) blending one of (i)-(iii) with the at least one of the substantially oxygen-free HFC-125, HFC-134a, HFC-134, HFC 152a, HFC-23, HCFO-Z/E-1233zd, and HFO-Z/E-1336mzz component.

26-30. (canceled)

31. A method, comprising passing a fluoroolefin composition containing at least HFO-1234yf and optionally (1) an oligomer/polymer inhibitor and/or (2) oxygen through one of:
a) silica gel or mineral oil at a temperature sufficient to remove the inhibitor; and
b) a column containing a reduced metal oxide wherein the metal oxide comprises at least one of Cu, Ti, V, Mn, Fe, Co, Zn, Ni and Pd as the metal and operating the column at ambient temperature to remove and/or reduce oxygen in the fluoroolefin composition to at most 10 ppm, preferably at most 5 ppm, without cleavage of the C—F bond of the fluoroolefin, and
c) recovering an HFO-1234yf-containing fluoroolefin containing at least one of less than 10 ppm, preferably less than 5 ppm oxygen, and less than 5 ppm oligomer inhibitor.

32. The method according to claim 31 wherein the fluoroolefin composition comprises at least HFO-1234yf and HFO-Z/E-1234ze, preferably wherein the fluoroolefin composition comprises about 0.1 and about 99.9 wt. % HFO-1234yf and about 0.1 and about 99.9 wt % HFO-Z/E-1234ze.

33. (canceled)

34. The method according to claim 31, wherein at least one additional component comprising one of HFC-125, HFC-134a, HFC-134, HFC 152a, HFC-23, HCFO-Z/E-1233zd, and HFO-Z/E-1336mzz contacts a column containing a reduced metal oxide operated at ambient temperature to provide a substantially oxygen-free HFC-125, HFC-134a, HFC-134, HFC 152a, HFC-23, HCFO-Z/E-1233zd, and HFO-Z/E-1336mzz component; and blending the substantially oxygen-free HFC-125, HFC-134a, HFC-134, HFC-23, HCFO-Z/E-1233zd, and HFO-Z/E-1336mzz additional component with the HFO-1234yf containing fluoroolefin containing at least one of less than 10 ppm, preferably less than 5 ppm, oxygen and less than 5 ppm oligomer inhibitor.

35. The method according to claim 31, further comprising a step of packaging the blend of claim 31.

36-38. (canceled)

39. The process as in claim 1, wherein one of said feed and fluoroolefin composition comprises unreclaimed refrigerant.

40-44. (canceled)
